(19) United States
(12) Patent Application Publication
(10) Pub. No.: US 2012/0128680 A1
(43) Pub. Date: May 24, 2012

(54) BIOMARKER ANTIBODY AND DIAGNOSIS DEVICE FOR DETECTING CERTAIN AUTOIMMUNE DISEASES

(75) Inventors: Maan Zrein, Ecully (FR); Philippe Vanhems, Sainte Foy Les Lyon (FR)

(73) Assignees: INFYNITY BIOMARKERS, Ecully (FR); CENTRE NATIONAL DE LA RECHERCHE SCIENTIFIQUE, Paris cedex 16 (FR); UNIVERSITE CLAUDE BERNARD LYON I, Villeurbanne cedex (FR); HOSPICES CIVILS DE LYON, Lyon cedex 02 (FR)

(21) Appl. No.: 13/319,207

(22) PCT Filed: May 6, 2010
(86) PCT No.: PCT/FR10/00349

§ 371 Date: Dec. 14, 2011

(30) Foreign Application Priority Data
May 7, 2009 (FR) 09/02240
Jun. 23, 2009 (FR) 09/03037

(51) Int. Cl. A61K 39/395; C07K 7/08; A61P 31/00; C12Q 1/70; G01N 33/569; C07K 16/44; G01N 33/566

(52) U.S. Cl. 424/139.1; 530/387.9; 530/326; 435/7.92; 435/5; 435/7.2

(57) ABSTRACT

Antibodies which specifically bind to a peptide having the sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi.

### CROSS-REFERENCE TO RELATED APPLICATIONS

The present application is a National Stage Application of PCT International Application No. PCT/FR2010/000349 (filed on May 6, 2010), under 35 U.S.C. §371, which claims priority to French Patent Application No. 0903037 (filed on Jun. 23, 2009) and French Patent Application No. 0902240 (filed on May 7, 2009), which are each hereby incorporated by reference in their respective entireties.

### FIELD OF THE INVENTION

The invention relates to the field of immunology, namely the field of interaction between an antigen and an antibody. More specifically, the invention relates to the use of a synthetic antigen (TCSP), derived from a protein of the unicellular parasite Trypanosoma Cruzi, which is the cause of Chagas disease, for the detection of antibodies not related to Chagas disease, which make it possible to diagnose certain autoimmune diseases in a human patient.

### BACKGROUND OF THE INVENTION

It is known that infectious agents can escape the immune system of a host by interfering with the normal maturation of an effective humoral immune response, and by directing induced antibodies against autoantigens. In addition, due to structural similarities between certain infectious antigens and self-antigens, it is thought that certain infectious agents are capable of triggering an autoimmune response in hosts that have a genetic predisposition.

Chagas disease is endemic in Latin America and South America, and is a major cause of morbidity and mortality in the countries where it flourishes. It is practically absent on other continents. Around 16 to 18 million people are infected, and around 50,000 patients die of it each year. It is the infection by the unicellular parasite Trypanosoma Cruzi (T. cruzi), a member of the Kinetoplastida order and the Trypanosomatidae family that induces Chagas disease in human beings; this protozoan parasite is transmitted by numerous hematophagous insects, and in particular by Triatominae (hematophagous plant bugs). Transmission takes place when the infectious forms of the parasite are deposited in an insect sting, when dejections of the vector insect come into contact with the blood or mucous membranes of the host. The vector insect thus releases infectious metacyclic trypomastigote forms that, by blood circulation, will colonize numerous cell types. The complex life cycle of the parasite enables it to escape the immune response of the host, without causing the death of the host. The parasite goes through an epimastigote step in the vector insect, a trypomastigote step in the blood of the host mammal, and an intracellular amastigote step: during this final step, the parasite multiplies by binary division. Another route of contamination is blood transfusion with contaminated blood; numerous screening methods have been developed (See, for example, EP Patent No. 0 976 763 and U.S. Pat. No. 6,458,922).

It is known that T. cruzi infects cardiac and skeletal muscle cells, glial cells and the cells of the mononuclear phagocyte system. After passive penetration in the host cell, the trimastigote form of the parasite is differentiated into the amastigote form; it actively divides, then the trypomastigote forms are released, thus causing a new cell invasion. Around 30% of patients with this disease develop severe clinical cardiomyopathy-type symptoms (See the article of K. Karratolios et al., “Inflammatory Cardiomyopathy”, published in 2006 in the review Hellenic Journal of Cardiology, vol. 47, p. 54-65, and the article of J. Burian et al., “Myocarditis: the immunologist's view on pathogenesis and treatment”, published in 2005 in the review Swiss Medical Weakly, vol. 135, p. 359-364); these cardiomyopathies may be acute or chronic.

Comparable cardiac conditions may be observed in patients infected by the human immunodeficiency virus (HIV) or other infectious agents outside of any Chagas disease context. Comparable cardiac conditions may also exist to a lesser extent in apparently healthy subjects; it is known (See, the article of F. Kierszenbaum, “Views on the autoimmunity hypothesis for Chagas disease pathogenesis”, published in 2003 in the review “FEMS Immunology and Medical Microbiology”, vol. 1545, p. 1-11, and the article of R. Jahns et al., “Pathological autoantibodies in cardiomyopathy”, published in September 2008 in the review Autoimmunity, vol. 41(6), p. 454-461) that these conditions result from autoimmune mechanisms of unknown origin. Indeed, autoimmune reactions can be observed in patients with cardiomyopathies without the precise origin, or the specificity of the antibodies, or especially of the immunogens involved, being known. No document describes a defined antigen structure, responsible for this autoimmunity and more specifically a T. cruzi (TCSP) antigen. Antibodies against receptors (adrenergic or cholinergic) have been described, but without any definition of their specificity with respect to the TCSP peptide; and in no way has the involvement of an antigen of the T. cruzi parasite outside of its natural context of Chagas disease been described.

It is known that each step of the life cycle of the T. cruzi parasite expresses specific antigen proteins, as described, for example, in the article of Hoft et al. (“Trypanosama cruzi Expresses Diverse Repetitive Protein Antigens”), published in July 1989 in the review Infection and Immunity, vol. 57 (7), p. 1959-1967). In the studies described in this publication, the authors screened a T. cruzi expression bank with human anti-serums and found cDNA that code for polypeptides containing repetitive units including 6 to 34 amino acids. The amino acid sequence of TCR70 (which is identical to TCR69) was strongly preserved, with only some occasional substitutions. Their frequent appearance in all of the isolated fractions and the diversity of these repetitive units suggest that they are involved in the circumvention of the destructive role of the immune system. Since these repetitive units are effective modulators of the immune system, it can be thought that other infectious agents use similar strategies, or even the same repetitive units.

The methods for screening antibodies directed against T. cruzi, which are used in blood banks (in particular in Brazil, where this screening is obligatory), include indirect immunofluorescence (IFA), indirect hemagglutination (IHA), and immunoenzymatic assay techniques (ELISA). Most of these assay kits, which are available on the market, use raw parasite extracts or sub-cellular fractions as antigen preparations. It was found that the parasite extracts react with serums of patients who have other diseases, such as leishmaniasis, the Trypanosoma rangeli infection, syphilis or rheumatoid fever. Consequently, the use of similar repetitive units by different pathogenic organisms complicates the diagnosis and treatment. In addition, a false positive response in the screening for Chagas diseases can lead to a false diagnosis that will be followed by a superfluous and/or ineffective treatment.

Consequently, there is an urgent need to be capable of detecting repetitive units that can be used by different pathogenic organisms. However, the prior art does not contain any documents showing or suggesting that the polypeptide motifs TCR70 identified in T. cruzi exist in other diseases unrelated to Chagas disease, or lead to autoimmune disorders. It has also not been shown or suggested in the prior art that these repetitive units can enable the development of reliable tools for the diagnosis and treatment or effective prevention of cardiac pathologies or autoimmune disorders associated or not with Chagas disease. There is therefore also a need to provide immunodiagnostic methods, which are preferably quick or capable of being used in the form of kits, for detecting pathogenic antibodies capable of binding specifically to the antigens expressed by T. cruzi. And finally, there are no methods for reducing the concentration of these pathogenic antibodies in the blood.

### SUMMARY OF THE INVENTION

In accordance with the invention, the stated problems are solved by the use of antibodies that bind specifically to a peptide including the sequence: Ala-Ala-Ala-Pro-Ala-Lys-Ala-Ala-Ala-Ala-Pro-Ala-Lys-Thr-A la-Ala-Ala-Pro-Val (SEQ ID N^{o}1), and which are not induced in a host after its infection by T. cruzi. These antibodies as such represent the first subject matter of this invention.

The second subject matter of the invention is an antigen that binds specifically to the antibody according to the first subject matter, with the exception of the antigens that bind also to antibodies induced by T. cruzi and which are genetically coded by the T. cruzi parasite, i.e. with the exception of antigens including the sequence SEQ ID N^{o}1.

The third subject matter is the use of a peptide or a protein including the sequence: Ala-Ala-Pro-Ala-Lys-Ala (SEQ ID N^{o}2), and preferably the sequence: Ala-Ala-Ala-Pro-Ala-Lys-Ala (SEQ ID N^{o}3), and even more preferably the sequence SEQ ID N^{o}1, for the detection and/or precipitation of antibodies according to the first subject matter, insofar as the peptide or the protein has a specific activity with respect to the antibodies. In particular, the peptide or the protein can be a TCSP peptide or a variant of the TCSP peptide, insofar as the variant has a specific activity with respect to the antibodies according to the first subject matter of the invention.

The antibodies can bind to antigens of an infectious agent, an allergen and/or a self-antigen; the infectious agent can be a pathogenic agent, and the presence of the antibodies is then associated with an infectious or autoimmune disease. The pathogenic agent can also be selected from the group formed by prions, viruses, prokaryotes and eukaryotes that are unicellular or multicellular.

The infectious or autoimmune disease can be selected from the group formed by cardiomyopathy, myocarditis and systemic lupus erythematosus.

Another subject matter of the invention is a method for detecting antibodies in accordance with the first subject matter in a liquid sample, which method includes the following steps: (a) placing a biological sample, preferably a sample of body fluid and/or supernatant liquid of a cellular culture (“liquid sample”), in contact with a peptide or a protein including the sequence SEQ ID N^{o}1, SEQ ID N^{o}2, SEQ ID N^{o}3, or with a TCSP peptide or a variant of the TCSP peptide, insofar as the peptide or the protein has a specific activity with respect to the antibodies in accordance with the invention; and then (b) determining the bond of the antibody in accordance with the invention in the liquid sample with the peptide or the protein by means of one or more suitable markers, capable of determining the complex formed between the peptide or protein and the antibody in accordance with the invention.

The marker can be selected from the group consisting of chemiluminescence markers, agglutination markers, membrane markers, immunoenzymatic markers and radioactive markers.

Yet another subject matter of the invention is a kit or a device for implementing the method in accordance with the previous subject matter, including: (a) a determined amount of one or more peptides and/or proteins the sequence SEQ ID N^{o}1, SEQ ID N^{o}2, SEQ ID N^{o}3, and/or of a TCSP peptide or a variant of the TCSP peptide, insofar as the peptides or the proteins has a specific activity with respect to the antibodies in accordance with the first subject matter of the invention; (b) one or more suitable markers, capable of determining the complex formed between the peptide or protein and the antibody according to the first subject matter of the invention; (c) optionally, a solid support, preferably impregnated by the peptide or the protein.

The marker can be selected from the group consisting of chemiluminescence markers, agglutination markers, membrane markers, immunoenzymatic markers and radioactive markers.

Yet another subject matter is an immunodiagnostic method including a step of qualitative or quantitative detection of the antibodies according to the first subject matter by the use of a peptide or a protein including the sequence SEQ ID N^{o}1, SEQ ID N^{o}2, SEQ ID N^{o}3, or with a TCSP peptide or a variant of the TCSP peptide, insofar as the peptide or the protein has a specific activity with respect to the antibodies in accordance with the first subject matter of the invention.

Yet another subject matter is a method for reducing the concentration of antibodies in accordance with the first subject matter in a biological fluid sample, such as a blood sample, or in a biological fluid flow, such as a blood fluid, including a step of precipitation of the antibodies by means of a peptide or a protein including the sequence SEQ ID N^{o}1, SEQ ID N^{o}2, SEQ ID N^{o}3, or with a TCSP peptide or a variant of the TCSP peptide, insofar as the peptide or the protein has a specific activity with respect to the antibodies in accordance with the first subject matter of the invention.

It is also possible to use the TCSP peptide or variants thereof to obtain antibodies or fragments of antibodies having a bonding activity equivalent to the NCRA (Non-Cruzi-Related Antibody, i.e. an antibody not induced by T. cruzi); this is yet another subject matter of the invention.

The peptides or proteins including one of the peptide sequences (SEQ ID N^{o}1, SEQ ID N^{o}2, SEQ ID N^{o}3) can be used to identify or even isolate the immunogens that induce the NCRA antibodies. Methods for conducting computer searches for sequence homologies using advanced algorithms can serve as tools for identifying immunogenic candidates. The immunogenic candidates can be synthesized, then tested for their reactivities with biological samples suspected of containing NCRA. The use of a peptide or a protein including the sequence SEQ ID N^{o}1, SEQ ID N^{o}2, SEQ ID N^{o}3 or of a TCSP peptide or a variant of the TCSP peptide, insofar as the peptide or the protein has a specific activity with respect to the antibodies according to the first subject matter of the invention, for identifying or isolating a pathogenic agent that binds specifically to the NCRA or that is recognized specifically by NCRA, forms another subject matter of the present invention. This interaction is based on a homology between the pathogenic agent and the TCSP or the variant of the latter.

Yet another subject matter of the invention is represented by the use of a peptide or a protein including the sequence SEQ ID N^{o}1, SEQ ID N^{o}2, SEQ ID N^{o}3 or of a TCSP peptide or a variant of the TCSP peptide, insofar as the peptide or the protein has a specific activity with respect to the antibodies in accordance with the first subject matter of the invention, for obtaining antibodies or antibody fragments having a bonding activity equivalent to NCRA.

The final subject matter of the invention is a pharmaceutical preparation including antibodies or antibody fragments having a bonding activity equivalent to NCRA, a pharmaceutically acceptable injectable solution and optionally one or more carriers (such as polysorbate and/or saccharose) or additives, in which the antibodies or antibody fragments have been obtained either by a use in accordance with the third subject matter of the invention in a living organism, leading to the immunization of the organism against the TCSP peptide, or by screening using the TCSP on a bank of bacteriophages that express specific antibodies.

### BRIEF DESCRIPTION OF THE DRAWINGS

FIG. 1 shows the distribution of the intensity of a signal associated with the presence of antibodies that bind specifically to the TCSP peptide, and which are not necessarily associated with a T. cruzi infection (these antibodies are called NCRA, Non-Cruzi-Related Antibodies). This data comes from an epidemiological trial involving a number N of patients belonging to four groups: DS=Healthy donors [French: donneurs sains] (i.e. blood donors monitored and selected, who therefore have a better health status than the average general population); HIV Africa: African patients with a positive response to an HIV virus screening; HIV Western: Western patient with a positive response to an HIV virus screening; Chagas: Patients infected by T. cruzi. The data contained in this table comes from ELISA-type assays. It is represented numerically in Tables 1 and 2.

FIG. 2 shows the results of an epidemiological study on 79 patients, namely the NCRA concentration as a function of time (in years) since the inclusion of the patients in the study.

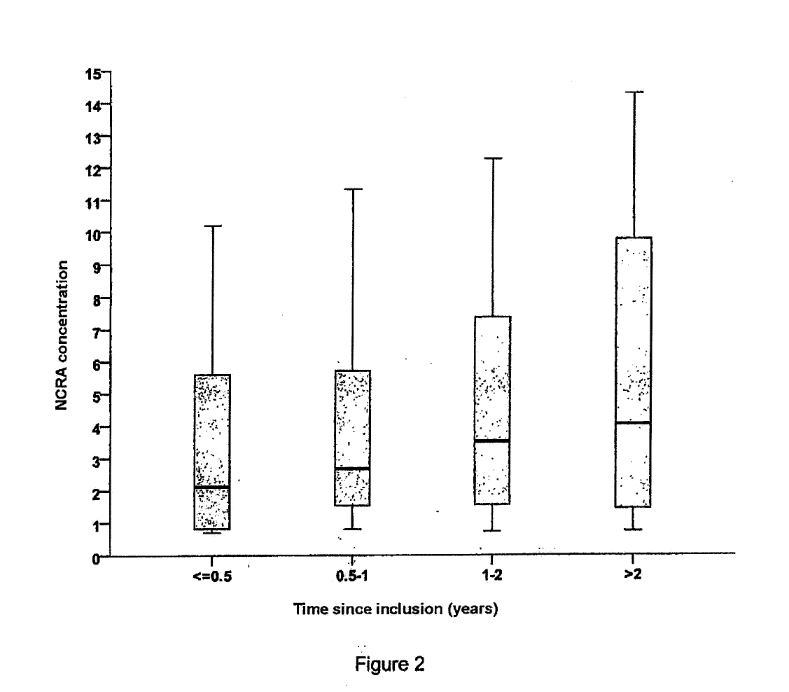

### DETAILED DESCRIPTION OF EMBODIMENTS

In the context of the invention, the following terms have a specific meaning. The term “Chagas disease” refers to a pathological state caused by infection with the Trypanosoma Cruzi parasite, via the parenteral route or not. The term “disease other than Chagas” refers to any pathological state not caused by the Trypanosoma Cruzi parasite, and which leads to increased reactivity of antibodies against the new antigen, referred to here as TCSP, defined hereinbelow.

By “amino acid,” it is meant natural and unnatural amino acids. “Natural” amino acids include the L form of amino acids that can be found in proteins of natural origin, i.e.: alanine (A), arginine (R), asparagine (N), aspartic acid (D), cysteine (C), glutamine (Q), glutamic acid (E), glycine (G), histidine (H), isoleucine (I), leucine (L), lysine (K), methionine (M), phenylalanine (F), proline (P), serine (S), threonine (T), tryptophane (W), tyrosine (Y) and valine (V).

“Unnatural” amino acids include the D form of natural amino acids, the homo forms of certain natural amino acids, such as: arginine, lysine, phenylalanine and serine, and the nor forms of leucine and valine. They also include other synthetic amino acids.

“Peptide compounds” include in particular peptides and polypeptides, including derivatives obtained for example by glycosylation, acetylation, phosphorylation, or reaction with fatty acids. This term also includes proteins and peptides of natural origin.

The term “antibody” includes polyclonal and monoclonal antibodies. The term “monoclonal antibody” refers to an antibody composition having a homogeneous population, regardless of the species, the origin and the method for obtaining said antibody. In addition, the term “antibody” includes human antibodies in which at least some of the immunoglobulin domains are present, such as antibody fragments and the so-called VHVL domains (Variable Heavy and Variable Light Chains), and mini-antibodies.

The term “NCRA” or “NCRA antibody” (Non-Cruzi-Related Antibodies) refers to antibodies that bind specifically to the TCSP antigen, and which are not induced in a host after its infection by T. cruzi.

The terms “TCSP” (Trypanosoma Cruzi Synthetic Peptide), “TCSP antigen”, “TCSP peptide” or “TCSP protein” all refer to a new peptide as defined by the invention, which comprises an amino acid sequence that is also found in proteins TCR70 and TCR69, namely SEQ ID N^{o}1, defined below, which can be isolated in T. cruzi, and which acts as an antigen with NCRA, as well as all variants and functional equivalents, such as its linear or non-linear epitopes recognized by NCRA as defined above. The minimal structure of the epitope corresponds to SEQ ID N^{o}2, defined below.

The term “self-antigen” refers to an epitope present in an endogenous molecule of the host, and which can be recognized by the immune system of said host, in order to possibly trigger an immune response. This mechanism can lead to an “autoimmune disease”, defined here as a pathological state caused by an undesired immune response of a host against a self-antigen (also called self epitope).

The term “infectious agent” refers here to any agent, living or not, capable of triggering an immune response. More specifically, it refers to pathogenic agents, allergens and haptens. “Pathogenic agents” include in particular prions, viruses, prokaryotes and eukaryotes that are isolated or not. “Allergens” include all substances or molecules capable of spontaneously causing an immune response in a host when said host is exposed to said substances or molecules.

The term “biological fluid” refers to the body fluid of a living organism, such as a human patient, i.e. any fluid sampled from a patient, such as serum, plasma, total blood, urine, cerebrospinal fluid, saliva, or the supernatant liquid of a cell culture.

In accordance with the invention, the problem is solved by the use of a new antibody that reacts with a new antigen (TCSP), derived from a protein of a unicellular parasite causing Chagas disease, Trypanosome Cruzi (T. cruzi). The particularity of this invention lies in the specific recognition of the TCSP antigen by antibodies not related to the T. cruzi parasite (called NCRA antibodies), therefore not having been induced by said antigen. This heterologous recognition phenomena is explained by the structural mimicry of the TCSP antigen with another immunogen, endogenous or exogenous, having immunized the individual and induced the NCRA.

The peptide sequence of the TCSP antigen is derived from a protein known and present in the databases Swissprot, Uniprot and TrEMBL (Accession Q7M3W1). This protein is known as TCR69; it is similar to protein TCR70.

In accordance with the invention, the peptide sequence of the protein used to define the NCRA antibody, expressed in three-letter amino acid codes, is:

(SEQ ID N^{o} 1)
Ala-Ala-Ala-Pro-Ala-Lys-Ala-Ala-Ala-Ala-Pro-Ala-
Lys-Thr-Ala-Ala-Ala-Pro-Val.

The immunoreactive variants of this peptide are also included in the context of this invention, insofar as they have the same antigenic properties. The variants of a peptide sequence can be obtained, for example, by substitution of one or more amino acids by other chemical entities, on the condition that the bioactivity of the original sequence is preserved; they can also be obtained by addition of chemical compounds such as biotin or natural or synthetic polymers, such as polylysine or polysaccharide. All of these variants that preserve the bioactivity of the original sequences are covered by this invention.

These variants can in particular be constituted by the sequence:

(SEQ ID N^{o} 2)
Ala-Ala-Pro-Ala-Lys-Ala, and preferably the
sequence:
(SEQ ID N^{o} 3)
Ala-Ala-Pro-Ala-Lys-Ala.

This sequence can be repeated one or more times, and during this repetition, the terminal unit (in the “C-terminal” sense) of alanine can be substituted by a threonine unit, as in SEQ ID N1. The peptides constituted by these sequences also have this same bioactivity and can be used in the context of this invention.

TCSP can be obtained by purification from the T. cruzi parasite protein extract, typically leading to proteins TCR69 or TCR70 (see the publication of Hoft et al., cited hereinabove), which have sequences SEQ ID N^{o}1, SEQ ID N^{o}2 and SEQ ID N^{o}3. TCSP and its variants can also result from chemical synthesis (for example, in accordance with the Merrifield method, which is well known to a person skilled in the art). They can also be obtained by molecular cloning technology, such as recombinant DNA, involving the protein expression in microbial expression systems after insertion of a nucleotide sequence coding for the sequence of the peptide, followed by culture, extraction and purification of the peptide of interest.

TCSP antigens and variants thereof cannot be used for screening for Chagas disease because they lead to false positive reactions. The inventors discovered that TCSP interacts specifically with NCRA. This means that TCSP comprises a specific peptide sequence, which is capable of binding to NCRA. This bond can be detected by any known method, such as chemiluminescence, agglutination methods, and immunoenzymatic or radioactive methods.

The biomarker antibody is present in biological fluids, and its presence is correlated with clinical symptoms in patients with cardiomyopathy, for example, subjects infected by HIV who have developed cardiac complications. This invention relates to the peptide sequence of TCSP as well as any structural analogue capable of binding to the same biomarker antibody and which are not necessarily induced by the T. cruzi parasite (NCRA). The invention also relates to an immunological assay method, for the detection and monitoring or myocarditis and cardiomyopathies in patients after an infection or autoimmune inflammation.

One embodiment of the invention is a method for determining, qualitatively or quantitatively, the NCRA concentrations in a biological sample, including the following steps: a) obtaining a biological sample, such as serum, plasma, total blood, urine, cerebrospinal fluid, saliva, a biopsy sample, or the supernatant liquid of a cell culture; and then b) determining the NCRA concentration in said biological sample.

The biological sample can be in liquid, gel or solid form. It can come from a patient or in vitro cultures.

The invention also relates to the monitoring of the treatment of cardiomyopathies, by determination of the NCRA concentration. Indeed, the method described above can also be applied to the estimation of the probability of fetal death caused by an in utero cardiac arrest, due to the presence of NCRA in pregnant women. Indeed, the transplacental passage of these antibodies can damage the cardiac cells during embryogenesis. Their effects are even more significant insofar as the cardiac tissue is primitive. In this method, a sample of a biological fluid of the patient, a pregnant woman, is obtained, and the NCRA concentration in this sample is determined.

In addition, the NCRA assay can lead to a therapeutic strategy in patients with cardiomyopathies or to the development of a vaccine against this disease. At present, the nature of the immunogen inducing the biomarker antibodies is not known; however, the TCSP sequence disclosed in this invention can lead to the characterization of the etiology of the disease, to the isolation of an infectious or non-infectious agent inducing said NCRA in human subjects not having been in contact with the T. cruzi parasite, and thus, contribute to the development of new therapeutic strategies.

As an example, antibodies or anti-TCSP antibody fragments can be designed so as to prevent, by competition, the binding of NCRA to their biological target site and thus inhibit their pathogenic effects. The design of such competitive antibodies is made possible by this discovery.

In addition, the longitudinal measurement (in the same patient over time) of NCRA can indicate a change in the cardiomyopathy and the efficacy of any treatment thereof. The subtraction of this same NCRA from the blood circulation, i.e., by immunoadsorption techniques, can reduce or even entirely suppress their pathogenic effects.

The invention is based on the surprising discovery of the antigenic properties of a protein isolated from the T. cruzi parasite in subjects who have never been in contact with this parasite. Indeed, the TCSP antigen has an exceptional “heterospecific” activity with NCRA, widespread in living human subjects outside of the endemic regions of the parasite. It clearly involves a peptide mimicry mechanism; these antibodies induce false positive reactions in a test screening for Chagas disease.

This mechanism enables the TCSP peptide to bind specifically to the NCRA directed against self-antigens or against infectious agents other than T. cruzi. Consequently, one embodiment of the invention is directed to the use the TCSP polypeptide or its immunoreactive variants, for the detection of NCRA. These NCRA antibodies can also bind to self-antigens, or to antigens of infectious organisms with more or less affinity and specificity than those of the bond with TCSP.

More specifically, this invention provides a polypeptide as described hereinabove, for identifying or isolating an infectious or non-infectious agent inducing antibodies that in turn can cause an autoimmune disease. The invention also provides a polypeptide as described above, in which said infectious agent is a virus or a microbe relatively widespread among human subjects apparently in good health, and largely widespread in particular in subjects infected by HIV, such as mycoplasms and other opportunistic infectious agents. The invention also makes it possible to use a polypeptide as described above, in which said autoimmune disease is chosen from those described in cardiac pathologies such as cardiomyopathies and myocarditis. In addition, the invention provides a method for detecting antibodies against the TCSP polypeptide, by placing the TCSP or a variant in contact with the NCRA present in a biological fluid sample, and the detection of the NCRA bonds in the biological sample by methods known to a person skilled in the art.

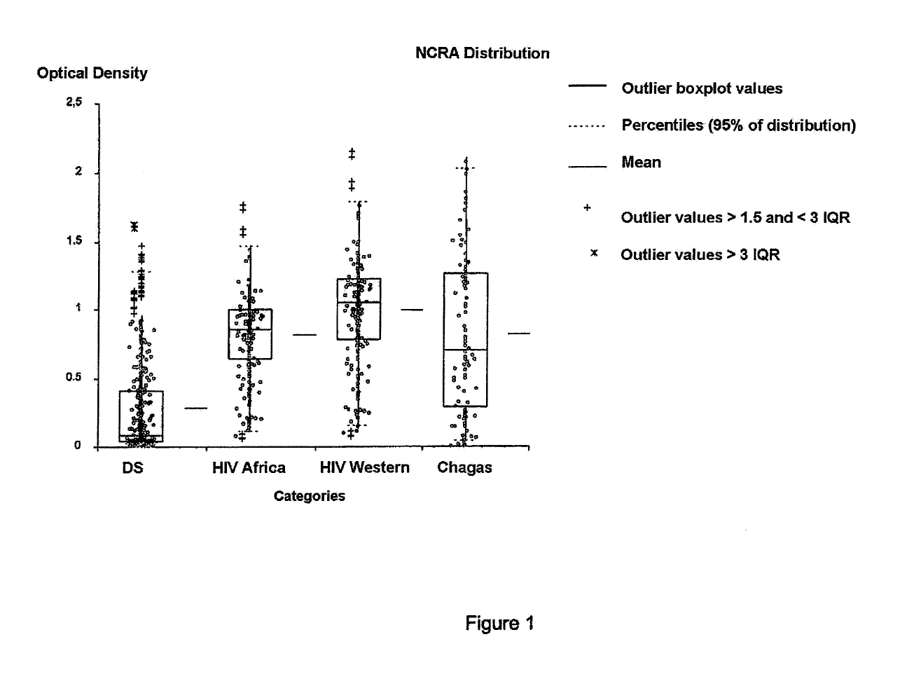

In addition, the invention provides an analysis method for the detection of antibodies against the TCSP polypeptide, including reagents or tools enabling the antigen-antibody bond to be detected by means of methods known to scientists in the field of immunoanalysis. In yet another embodiment of the invention, the TCSP polypeptide or its variants are used, insofar as the latter have a specific activity with respect to NCRA, to improve the specificity of serological screening analyses for Chagas disease. It is clear from the results shown in FIG. 1 that the NCRA, detected in screening tests, can have an origin other than an infection by the T. cruzi parasite. Consequently, the inhibition of these antibodies by the TCSP antigen can improve the specificity of Chagas disease diagnostic tests.

The immunoreactivity tests in this invention were performed by an ELISA (Enzyme-Linked Immunosorbent Assay) technique, known to a person skilled in the art. Any other technique, however, making it possible to detect or measure the antigen-antibody bond can also be applied to the invention, in particular in order to implement the new immunodiagnostic method that forms one of the subjects of the invention.

Another aspect of the invention is the therapeutic use of the TCSP peptide or its variants to obtain antibodies or antibody fragments having a bonding activity equivalent to NCRA. In this use, antibodies or antibody fragments are first prepared by immunization of a living organism (for example, animal) against the TCSP peptide. Then, a pharmaceutical preparation including these antibodies or antibody fragments, a pharmaceutically acceptable injectable solution and optionally adjuvants (such as polysorbate, saccharose) is prepared.

Then, this pharmaceutical preparation is administered (for example, injected) to a patient in order to compete with the pathogenic NCRA. Thus, the pathogenic NCRA concentration is reduced, and the clinical symptoms of the patient improve.

Yet another aspect of the invention is a method for reducing the concentration of antibodies in accordance with the invention in a biological fluid sample, and in particular a blood sample or in a blood flow, including a step of retaining the antibodies by means of a peptide or a protein including the sequence SEQ ID N^{o}1, SEQ ID N^{o}2, SEQ ID N^{o}3, or with a TCSP peptide or a TCSP peptide variant, insofar as said peptide or said protein has a specific activity with respect to the antibodies in accordance with the invention. This method can be implemented for the purpose of diagnosis, for the purpose of treatment of a quantity of biological fluid or for therapeutic purposes by plasmapheresis. It can be implemented statically or in a flow of the biological fluid. Preferably, it is implemented as an immunoadsorption method. In a typical embodiment, the biological fluid comes into contact with a solid support on which the peptide or the protein or the protein including sequence SEQ ID N^{o}1, SEQ ID N^{o}2, SEQ ID N^{o}3, or the TCSP peptide or a variant of the TCSP peptide is fixed. The antibodies, if present, then precipitate on this solid support and are removed from the biological fluid.

Another aspect of the invention is a pharmaceutical preparation including antibodies or antibody fragments having an activity equivalent to NCRA, a pharmaceutically acceptable injectable solution and optionally one or more adjuvants, such as polysorbate and/or saccharose. These antibodies or antibody fragments can be obtained in different ways. In one embodiment, they can be obtained by screening using TCSP on a bank of bacteriophages that express specific antibodies. In another embodiment, they can be obtained in a living organism, using a peptide or protein including sequence SEQ ID N^{o}1, SEQ ID N^{o}2, or SEQ ID N^{o}3 for the detection of antibodies that bind specifically to a peptide including sequence SEQ ID N1 and which are not induced in a host after its infection by T. cruzi, in which the use leads to the immunization of the organism against the TCSP peptide.

Yet another aspect of the invention is the use of TCSP, and in particular TCSP including sequence SEQ ID N^{o}1, SEQ ID N^{o}2 or SEQ ID N^{o}3, in a vaccine composition, making it possible to immunize a human at least partially against a disease other than Chagas disease, and in particular against autoimmune cardiomyopathy, myocarditis and systemic lupus erythematosus. This vaccination can be performed by injection of a single dose or a plurality of doses. In these compositions, the TCSP can be used as such, as a peptide, or in biotinylated form and/or linked to an avidin derivative, and in particular an avidin derivative obtained by chemical and enzymatic modification, known as NeutraLite Avidin. The composition can include solvents, additives, buffers and pharmaceutically acceptable carriers, as well as, as the case may be, other active principles.

In accordance with another aspect of the invention, a peptide or a protein including sequence SEQ ID N^{o}1, SEQ ID N^{o}2, SEQ ID N^{o}3, or a TCSP peptide or a variant of the TCSP peptide is used, insofar as the peptide or the protein has a specific activity with respect to the antibodies in accordance with the invention, in order to identify or isolate a pathogenic agent that binds specifically to NCRA or that is recognized specifically by NCRA. This new method makes it possible to identify the pathogenic agents capable of inducing diseases, other than Chagas disease, that induce the formation of NCRA.

In some of the uses of TCSP in accordance with the invention, it may be advantageous to mark the TCSP, for example by means of a chromophore or a fluorophore, or another chemical entity that can easily be detected (enzyme, radioactive isotope, biotin, hapten, etc.).

The following examples illustrate certain aspects of the invention, without limiting it.

### GROUP STUDY EXAMPLES

### Example 1

These examples correspond to screening tests performed on a number N of a plurality of human populations. Tables 1 and 2 as well as FIG. 1 show results of these screening tests. The ELISA diagnostic technique used for these tests, as well as the successive steps of the tests, are described here.

### ELISA Technique

The TCSP peptide, represented by a synthetic peptide of sequence SEQ ID N^{o}1, was adsorbed on polystyrene microplates with a concentration of 1 μg/m. The free attachment sites were then saturated by immunologically neutral proteins (in this case albumin). The microplates were then rinsed with a washing buffer, then dried so as to be ready for use. The samples to be tested were incubated in the wells after 1/20 dilution in a dilution buffer; the antibodies not fixed to the antigenic surface were removed by three washing cycles. The specific antibodies of the TCSP fixed to the microplates were detected by an antibody directed against the human IgG and marked by an enzymatic tracer. A chromogenic substrate of the enzyme used served to expose the attachment of the anti-TCSP by measuring the optical density corresponding to the absorption of the chromogen in an appropriate range of wavelengths, and in particular at a wavelength of around 450 nm.

Reaction of the TCSP peptide with the NCRA present in the serums obtained from patients infected by HIV.

The serum samples of patients infected by HIV were tested for the presence of NCRA, for two populations: African patients and Western patients (European and American sources).

Reaction of the TCSP peptide with serums obtained from “healthy” blood donors, i.e. negative for anti-HIV, anti-HVC, anti-HVB antibodies and syphilis.

To evaluate the prevalence of antibodies against TCSP, on the basis of the hypothesis that these antibodies were not induced by a T. cruzi infection, European serum samples were used. These serums were obtained by healthy blood donors, as indicated above; these donors are therefore in a better state of health than the average population from which they are obtained.

To be completely certain with regard to the origin of these antibodies against TCSP, all of the samples were tested in a random order using commercially available kits making it possible to detect a T. cruzi infection; no positive sample was found. However, and in a totally unexpected manner, it was found that a significant percentage of these samples nevertheless contained antibodies against TCSP. It is concluded that T. cruzi uses a highly immunogenic motif that may also be used by other pathogens, and/or that may lead to autoimmune antibodies.

Reaction of the TCSP peptide with the NCRA present in patient serums: by interacting serum samples and solid surfaces on which the TCSP has previously been adsorbed.

Reaction of the TCSP with the serums of patients infected by the T. cruzi parasite: Given that the TCSP antigen is derived from a protein of the T. cruzi parasite, it is difficult, by conventional techniques, to distinguish the antibodies induced by the T. cruzi infection from those induced by the autoimmunity mechanism (NCRA). In an ELISA test using the TCSP as an antigen, it was found that the two categories of antibodies are confused; the EIA result is not discriminant.

The results are documented in Tables 1 and 2 as well as in FIG. 1. FIG. 1 illustrates, for each population of N patients, the distribution of the reactivity (expressed as optical density) of NCRA with the TCSP peptide used for the trial. The distribution of individual points are represented for each of the populations; the rectangles (boxes) show the semi-interquartile ranges. The solid horizontal lines show the arithmetic mean of each of the populations, and the dotted horizontal lines define 95% of the population.

TABLE 1

| | | | Confidence | | | |
|---|---|---|---|---|---|---|
| Patient | Number | | interval 95% | | Standard | Standard |
| category | N | Average | (min/max) | | error | deviation |
| Healthy | 576 | 0.2833 | 0.2531 | 0.3134 | 0.01535 | 0.36843 |
| donors | | | | | | |
| African | 192 | 0.8167 | 0.7708 | 0.8627 | 0.02330 | 0.32281 |
| HIV | | | | | | |
| Western | 192 | 0.9976 | 0.9417 | 1.0535 | 0.02834 | 0.39274 |
| HIV | | | | | | |
| Chagas | 96 | 0.8228 | 0.7083 | 0.9372 | 0.05766 | 0.56490 |
| patients | | | | | | |

TABLE 2

| | | | | | Confidence | | | | |
|---|---|---|---|---|---|---|---|---|---|
| Patient | | Mini- | 1rst | Median | interval 95% | | 3rd | Maxi- | |
| category | N | value | quartile | value | (min/max) | | quartile | value | IQR |
| Healthy | 576 | 0.026 | 0.0400 | 0.0795 | 0.0640 | 0.0970 | 0.4076 | 1.614 | 0.3676 |
| donors | | | | | | | | | |
| African | 192 | 0.050 | 0.6393 | 0.8575 | 0.8200 | 0.9020 | 0.9988 | 1.762 | 0.3595 |
| HIV | | | | | | | | | |
| Western | 192 | 0.063 | 0.7808 | 1.0470 | 1.0000 | 1.1000 | 1.2193 | 2.150 | 0.4385 |
| HIV | | | | | | | | | |
| Chagas | 96 | 0.037 | 0.2873 | 0.7055 | 0.5950 | 0.9810 | 1.2640 | 2.111 | 0.9768 |
| patients | | | | | | | | | |

IQR (Interquartile range) means the semi-interquartile range

### Example 2

In another group of N=79 patients infected with HIV, a clear relationship between the increase in NCRA measurements and the chronological development of these patients on a time scale going beyond two years was noted (see FIG. 3).

This study shows that the monitoring of NCRA concentrations can enable the prognosis of the development the HIV infection and its consequences in terms of cardiac complications.

### Examples of Individual Cases (Clinical Trials)

In cross-studies, the NCRA concentration in male patients infected with HIV was measured, and it was possible to study their cardiac condition. Table 3 shows these results. As an example, for patient PAT_{—}001, the presence of a high NCRA assay (namely: 8.19) is noted in a blood sample taken prior to his cardiac event.

These examples show the relationship between the measured NCRA assays and subsequent cardiac events (see PAT_{—}001, PAT_{—}004, PAT_{—}005), and the possibility that a sub-clinical cardiac event can induce the appearance of NCRA at a high dose (see PAT_{—}009).

TABLE 3

| | | | | | | | Serum |
|---|---|---|---|---|---|---|---|
| | | HIV-1 | CD4 cells | Viral load | | NCRA | tested |
| Patient | Birth | CDC | [number/ | [Copies/ | Cardiological | Signal/ | (sample |
| number | year | stage | mm3] | mL] | clinical data | noise | date) |
| PAT_001 | 1944 | A2 | 1110 | 50 | Myocardial | 8.19 | Nov. 15, 2004 |
| | | (Dec. 20, 2005) | | | infarction | | |
| | | | | | Mar. 5, 2006 | | |
| PAT_004 | 1963 | B3 | 512.5 | 50 | Myocardial | 2.21 | Sep. 3, 2004 |
| | | (Dec. 3, 2002) | | | incident Mar. 7, 2005 | | |
| PAT_005 | 1957 | A3 | 333.7 | 40 | Myocardial | 2.56 | Sep. 9, 2008 |
| | | (Jun. 18, 2003) | | | infarction | | |
| | | | | | Dec. 14, 2008 | | |
| PAT_009 | 1945 | A3 | 488.8 | 50 | Infarction | 25.15 | Sep. 23, 2004 |
| | | (May 3, 1994) | | | without prior | | |
| | | | | | Q wave | | |
| | | | | | Nov. 3, 2000 | | |

1-17. (canceled)

18. Antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi.

19. An antigen that binds specifically to antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi,
wherein an antigen genetically coded by T. cruzi does not bind to the antibodies.

20. Use of one of a peptide and a protein comprising at least one of the following sequences:
Ala-Ala-Ala-Pro-Ala-Lys-Ala-Ala-Ala-Ala-Pro-Ala-Lys-Thr-Ala-Ala-Ala-Pro-Val (SEQ ID N^{o}1), Ala-Ala-Pro-Ala-Lys-Ala (SEQ ID N^{o}2) and Ala-Ala-Ala-Pro-Ala-Lys-Ala (SEQ ID N^{o}3), for at least one of a detection and a precipitation of antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi,
wherein one of the peptide and the protein has a specific activity with respect to the antibodies.

21. The use of claim 20, wherein:
one of the peptide and the protein is one of a TCSP peptide and a variant of the TCSP peptide; and
the variant of the TCSP peptide has a specific activity with respect to the antibodies.

22. The use of claim 20, wherein the antibodies bind to antigens of at least one of an infectious agent, an allergen and a self-antigen.

23. The use of claim 22, wherein:
the infectious agent is a pathogenic agent; and
the presence of the antibodies is associated with one of an infectious disease and an autoimmune disease.

24. The use of claim 23, wherein the pathogenic agent is selected from the group formed by prions, viruses, prokaryotes and eukaryotes that are one of unicellular and multicellular.

25. The use of claim 23, wherein one of the infectious disease and the autoimmune disease is selected from the group formed by cardiomyopathy, myocarditis and systemic lupus erythematosus.

26. A method for detecting in a liquid sample, antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi, the method comprising:
placing a biological sample in contact with one of:
(a) one of a peptide and a protein comprising the sequence SEQ ID N^{o}1, a sequence Ala-Ala-Pro-Ala-Lys-Ala (SEQ ID N^{o}2) and a sequence Ala-Ala-Ala-Pro-Ala-Lys-Ala (SEQ ID N^{o}3), wherein the peptide and the protein has a specific activity with respect to the antibodies; and
(b) one of a TCSP peptide and a variant of the TCSP peptide; and then determining a bond of the antibodies in the biological sample with one of the peptide and the protein via at least one marker configured to determine a complex formed between the antibodies and one of the peptide and the protein.

27. The method of claim 26, wherein the biological sample comprises a sample of body fluid.

28. The method of claim 26, wherein the biological sample comprises a supernatant liquid of a cellular culture.

29. The method of claim 26, wherein the marker is selected from the group consisting of chemiluminescence markers, agglutination markers, membrane markers, immunoenzymatic markers and radioactive markers.

30. A kit for implementing a method for detecting in a liquid sample, antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi, the kit comprising:
a predetermined amount of at least one of:
(a) at least one peptides and proteins comprising the sequence SEQ ID N^{o}1, a sequence Ala-Ala-Pro-Ala-Lys-Ala (SEQ ID N^{o}2) and a sequence Ala-Ala-Ala-Pro-Ala-Lys-Ala (SEQ ID N^{o}3), wherein the peptide and the protein has a specific activity with respect to the antibodies; and
(b) one of a TCSP peptide and a variant of the TCSP peptide;
at least one marker configured to determine a complex formed between the antibodies and one of the peptide and the protein; and
a support impregnated by one of the peptide and the protein.

31. The kit of claim 30, wherein the marker is selected from the group consisting of chemiluminescence markers, agglutination markers, membrane markers, immunoenzymatic markers and radioactive markers.

32. An immunodiagnostic method comprising:
detecting antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi, via one of:
(a) one of a peptide and a protein comprising the sequence SEQ ID N^{o}1, wherein the peptide and the protein has a specific activity with respect to the antibodies;
(b) one of a TCSP peptide and a variant of the TCSP peptide.

33. The immunodiagnostic method of claim 32, wherein the detecting of the antibodies comprises one of qualitative detecting and quantitative detecting.

34. A method for reducing in one of a biological sample and a biological fluid flow, a concentration of antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi, the method comprising:
precipitating the antibodies via one of:
(a) one of a peptide and a protein comprising the sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), wherein the peptide and the protein has a specific activity with respect to antibodies that bind specifically to a peptide comprising the sequence (SEQ ID N^{o}1), and which are not induced in a host following an infection of the host with T. cruzi;
(b) one of a TCSP peptide and a variant of the TCSP peptide.

35. A method of obtaining one of antibodies and antibody fragments having a bonding activity equivalent to NCRA, the method comprising:
using of one of:
(a) one of a peptide and a protein comprising the sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), wherein the peptide and the protein has a specific activity with respect to antibodies that bind specifically to apeptide comprising the sequence (SEQ ID N^{o}1), and which are not induced in a host following an infection of the host with T. cruzi;
(b) one of a TCSP peptide and a variant of the TCSP peptide.

36. A method to one of identify and isolate a pathogenic agent that one of binds specifically to NCRA and is recognized specifically by NCRA, the method comprising:
using one of:
(a) one of a peptide and a protein comprising the sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), wherein the peptide and the protein has a specific activity with respect to antibodies that bind specifically to a peptide comprising the sequence (SEQ ID N^{o}1), and which are not induced in a host following an infection of the host with T. cruzi;
(b) one of a TCSP peptide and a variant of the TCSP peptide.

37. A pharmaceutical preparation including one of antibodies and antibody fragments having a bonding activity equivalent to NCRA, a pharmaceutically acceptable injectable solution and at least one of a polysorbate carrier and a saccharose carrier or additives, in which one of said antibodies and antibody fragments have been obtained by a use in a living organism of one of a peptide and a protein comprising at least one of the following sequences:
Ala-Ala-Ala-Pro-Ala-Lys-Ala-Ala-Ala-Ala-Pro-Ala-Lys-Thr-Ala-Ala-Ala-Pro-Val (SEQ ID N^{o}1), Ala-Ala-Pro-Ala-Lys-Ala (SEQ ID N^{o}2) and Ala-Ala-Ala-Pro-Ala-Lys-Ala (SEQ ID N^{o}3), for at least one of a detection and a precipitation of antibodies that bind specifically to a peptide comprising a sequence Ala-Ala-Ala-Pro-Ala-Lys-Ala-Ala-Ala-Ala-Pro-Ala-Lys-Thr-Ala-Ala-Ala-Pro-Val (SEQ ID N^{o}1), and which are not induced in a host following an infection of the host with T. cruzi, wherein one of the peptide and the protein has a specific activity with respect to the antibodies, leading to the immunization of said organism against the TCSP peptide.


SEQUENCE LISTING (3 sequences)

SEQ ID NO: 1 - protein, 19 aa, Artificial
  Trypanosoma Cruzi Related Peptide
AAAPAKAAAAPAKTAAAPV

SEQ ID NO: 2 - protein, 6 aa, artificial
  Trypanosoma Cruzi Related Peptide
AAPAKA

SEQ ID NO: 3 - protein, 7 aa, artificial
  Trypanosoma Cruzi Related Peptide
AAAPAKA